(19) United States
(12) Patent Application Publication
(10) Pub. No.: US 2009/0023842 A1
(43) Pub. Date: Jan. 22, 2009

(54) PORPHYRIN AND CONDUCTIVE POLYMER COMPOSITIONS FOR USE IN SOLID-STATE ELECTRONIC DEVICES

(75) Inventors: Darin W. Laird, Pittsburgh, PA (US); Shawn P. Williams, Pittsburgh, PA (US); Elena E. Sheina, Pittsburgh, PA (US); Jonathan S. Lindsey, Raleigh, NC (US); Gregory N. Parsons, Raleigh, NC (US)

Correspondence Address: FOLEY AND LARDNER LLP;SUITE 500, 3000 K STREET NW, WASHINGTON, DC, 20007, US

(73) Assignees: Plextronics, Inc.; North Carolina State University

(21) Appl. No.: 12/124,972

(22) Filed: May 21, 2008

Related U.S. Application Data
(60) Provisional application No. 60/939,340, filed on May 21, 2007.

(51) Int. Cl. C08K 5/3417; C08G 75/00; C08L 81/00; H01L 31/00; C08K 3/04

(52) U.S. Cl. 524/90; 528/380; 524/609; 524/424; 136/252; 977/734

(57) ABSTRACT

Compositions comprising porphyrinic macrocycles and conjugated polymers such as polythiophene for use in organic electronic devices including solar cells are presented. Covalent linkage of a porphyrinic macrocycle to a polymer allows tuning of electronic and spectroscopic properties of conjugated polymers and can improve the heat stability of the system relative to a blended comparison. A composition comprising: at least one polymer comprising at least one porphyrinic macrocycle covalently linked to at least one conjugated polymer, wherein the porphyrinic macrocycle is metal-free is also presented. Inks can be formulated. Methods of making are provided.

### RELATED APPLICATIONS

This application claims benefit of U.S. provisional application Ser. No. 60/939,340 filed May 21, 2007, which is incorporated by reference in its entirety.

### BACKGROUND

Electronic and optical properties of inherently conductive or conjugated polymers can be tuned to improve the performance of polymer-based electronic devices, such as light emitting diodes, photovoltaic cells, and field effect transistors. These devices and materials are of interest in, for example, displays, off-grid power generation, and low weight, flexible, and printable circuitry. It is of great importance to improve the performance of currently existing devices including enhancing their efficiencies and tunability. Among the various families of conjugated polymers, polythiophenes, including regioregular polythiophenes, are particularly useful. See, for example, McCullough et al, U.S. Pat. Nos. 6,602,974 and 6,166,172, which are incorporated by reference in their entirety. See also Plextronics US Patent Publication 2006/0076050 to Williams et al., “Heteroatomic Regioregular Poly(3-Substitutedthiophenes) for Photovoltaic Cells.”

Also useful are porphyrinic pigments (e.g., porphyrins, chlorins, and bacteriochlorins) which exhibit intense absorption in the blue, red, and near infrared (NIR, 700-900 nm) region. Recent advances in the synthetic chemistry of porphyrinic materials, including chlorins and bacteriochlorins, now enable access to a wide range of analogues of the natural pigments. The synthetic pigments exhibit spectral and photophysical attributes similar to those of the natural pigments, but have advantages over the natural materials in terms of stability and synthetic variability. The latter permits facile tuning of spectral features, photophysical properties, redox potentials, and self-assembly or building block attributes. Thus, porphyrins are available that bear four distinct meso substituents. Stable chlorins are now available wherein control of substituents can be exercised at all but one site, enabling tuning of absorption from 605-685 nm. Stable bacteriochlorins are now available wherein distinct patterns of substituents can be introduced; the absorption spectrum can be tuned from 730-800 nm. Thus, synthetic porphyrinic pigments can be used to address fundamental and commercial questions regarding solar-energy transduction in molecular materials whose design is inspired by natural photosynthetic assemblies. Synthetic porphyrinic pigments have been used as light harvesting arrays, as disclosed in, for example, Lindsey et al., U.S. Pat. Nos. 6,420,648; 6,916,982; 6,596,935; 6,407,330; 6,603,070, herein incorporated by reference in their entirety.

A need exists to provide materials which comprise both porphyrins and polymers to satisfy sophisticated application demands. For example, better performance is needed in parameters such as, for example, work function, oxidation onset, efficiency, and open circuit voltage. In particular, better photovoltaic materials are needed including materials that are processable and stable. Moreover, more versatile synthetic strategies are needed.

Schaferling et al., J. Mater. Chem., 2004, 14, 1132-1141 illustrates the difficulty in synthetically combining a porphyrin and a conjugated polymer. They report making porphyrin-functionalized polythiophenes by electropolymerization, but polymerization could not occur without the metal present in the porphyrin and molecular weight data are not provided. Polymers formed by electropolymerization can be difficult to characterize and can yield undefined films.

### SUMMARY

Compositions, devices, methods of making, methods of using are provided herein.

One embodiment provides a composition comprising: at least one polymer comprising at least one porphyrinic macrocycle bonded to at least one conjugated polymer, wherein the porphyrinic macrocycle is metal-free.

Another embodiment provides a composition comprising: at least one polymer comprising at least one porphyrinic macrocycle covalently linked to at least one conjugated polymer, wherein the conjugated polymer has at least ten conjugated repeat units.

Another embodiment provides a composition prepared by: providing at least one porphyrinic macrocycle, providing at least one conjugated polymer, covalently linking the conjugated polymer and the porphyrinic macrocycle.

Another embodiment provides a method comprising: providing at least one porphyrinic macrocycle, providing at least one conjugated polymer, covalently linking the conjugated polymer and the porphyrinic macrocycle.

Another embodiment provides a composition comprising a blend of: at least one conjugated polymer, wherein the conjugated polymer comprises a polythiophene, at least one porphyrinic macrocycle, wherein the conjugated polymer and the porphyrinic macrocycle are not bonded to each other.

Another embodiment is a composition comprising a blend of: at least one p-type semiconductor and at least one additive which absorbs in the UV and IR outside of the absorption region of the semiconductor. For example, the semiconductor can be a conjugated polymer and the additive can be a porphyrinic macrocycle.

Advantages in one or more embodiments include, for example, increased use of the solar spectrum for electrical current generation, better efficiency, synthetic versatility, ability to tune the energetics of the system components, and preservation of absorption bands like soret band at 420 nm despite annealing. In one embodiment, covalent linkage of a porphyrinic macrocycle to a polymer can improve the heat stability of the system relative to a blended comparison.

### BRIEF DESCRIPTION OF THE DRAWINGS

FIG. 1 shows an example of a photovoltaic or solar cell.

FIG. 2 shows schematic representations of porphyrinic macrocycle-polymer conjugates.

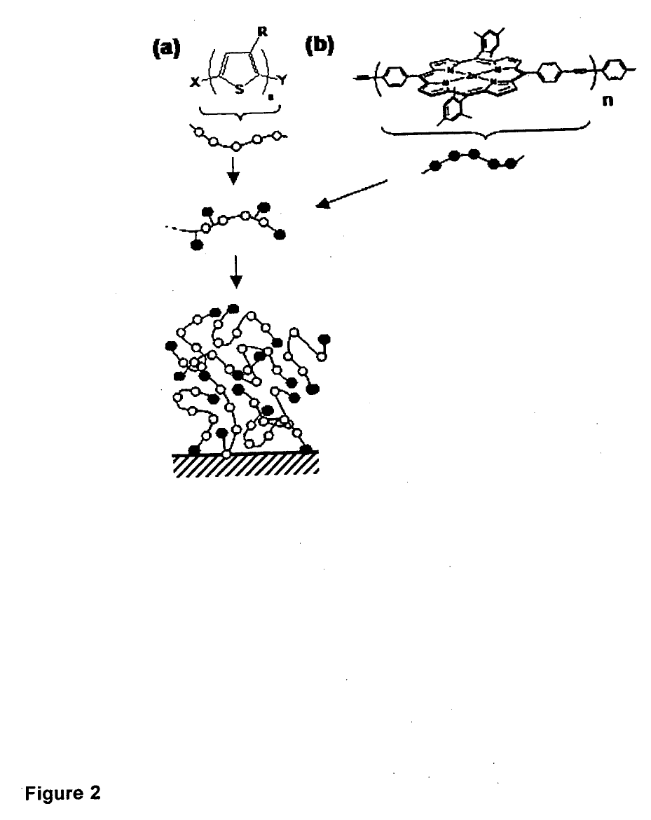

FIG. 3 shows compounds used to prepare porphyrinic macrocycle-polymer conjugates of preferred embodiments and different points of attachment of porphyrinic macrocycle with polymer.

FIG. 4 shows the SEC trace of the crude reaction mixture upon Sonogashira coupling of H—Br terminated P3HT (P2) and a diethynylporphyrin (upper panel), and the mixture of starting materials (lower panel). The wavelength of detection was 520 nm. The absorption spectrum of each component eluting in advance of the P3HT polymer (upper panel) showed the characteristic absorption peaks of both the porphyrin and the polymer. These data pertain to reaction VII.

FIG. 5 shows absorption spectrum of the crude reaction mixture upon Sonogashira coupling of H—Br terminated P3HT (P2) and a diethynylporphyrin in CH₂Cl₂/ethanol (3:1) at room temperature. The absorption spectrum of the product obtained upon preparative SEC is identical with that displayed here. The characteristic band of the porphyrin (422 nm) and those of the P3HT polymer (500-600 nm region) are clearly visible. These data pertain to reaction VII.

### DETAILED DESCRIPTION OF THE PREFERRED EMBODIMENTS

### Introduction

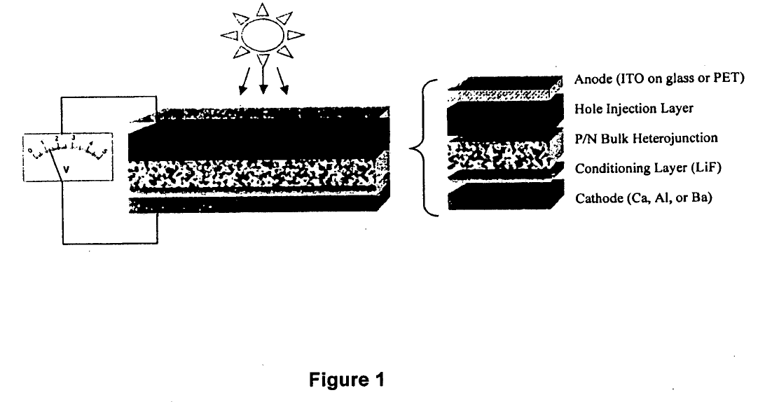

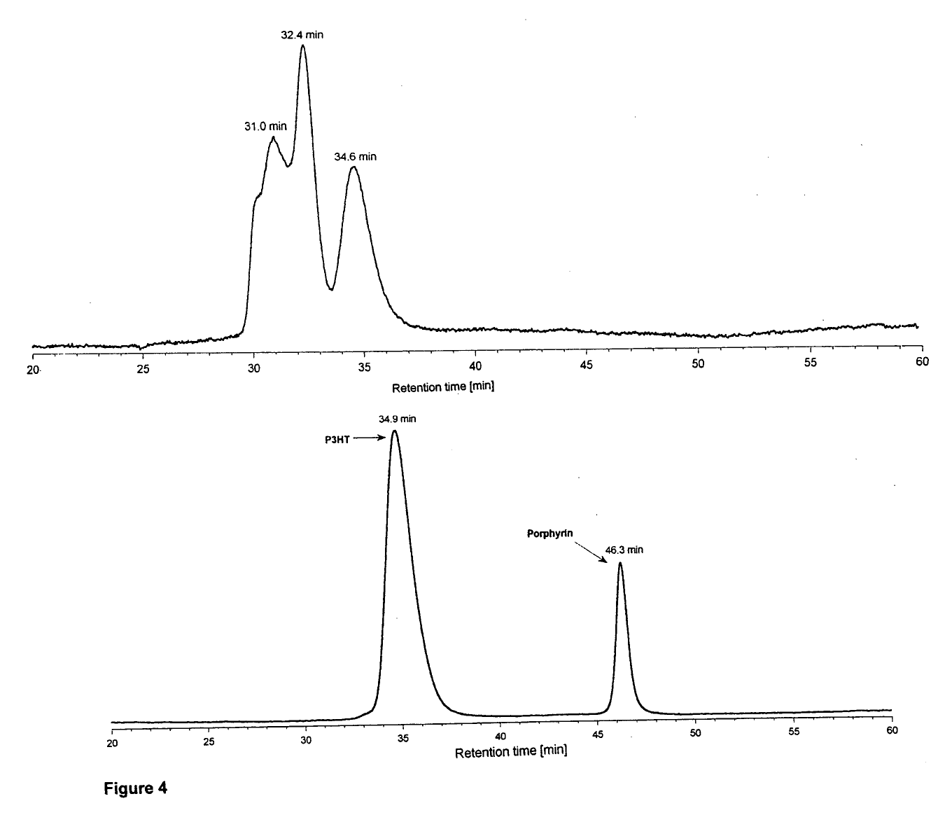

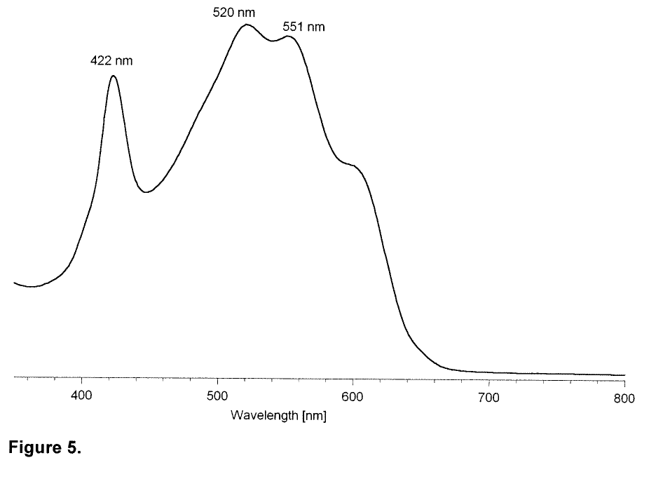

FIG. 1 illustrates some components of a conventional solar cell. See also for example Dennler et al., “Flexible Conjugated Polymer-Based Plastic Solar Cells: From Basics to Applications,” Proceedings of the IEEE, vol. 93, no. 8, August 2005, 1429-1439, including FIGS. 4 and 5. Various architectures for the solar cell can be used. Important elements include the active layer, an anode, a cathode, and a substrate to support the larger structure. In addition, a hole injection layer and/or hole transport layer can be used, and a conditioning layer can be used. The active layer can comprise a P/N composite including for example a P/N bulk heterojunction.

The following references describe photovoltaic materials and devices:

US Patent Publication 2006/0076050 to Williams et al., “Heteroatomic Regioregular Poly(3-Substitutedthiophenes) for Photovoltaic Cells,” (Plextronics) which is hereby incorporated by reference in its entirety including working examples and drawings.

US Patent Publication 2006/0237695 (Plextronics), “Copolymers of Soluble Poly(thiophenes) with Improved Electronic Performance,” which is hereby incorporated by reference in its entirety including working examples and drawings.

In addition, US Patent Publication 2006/0175582 “Hole Injection/Transport Layer Compositions and Devices” describes hole injection layer technology, (Plextronics) which is hereby incorporated by reference in its entirety including working examples and drawings.

In addition, U.S. patent application Ser. No. 11/743,587 filed May 2, 2007 describes active layer compositions and solar cell devices and is hereby incorporated by reference in its entirety.

In addition, U.S. patent application Ser. No. 12/113,058 filed Apr. 30, 2008 describes active layer compositions and processing methods and solar cell devices and is hereby incorporated by reference in its entirety.

In addition, U.S. patent application Ser. No. 11/826,394 filed Jul. 13, 2007 describes hole injection layer compositions and solar cell and other organic electronic devices and is hereby incorporated by reference in its entirety.

U.S. Pat. No. 7,147,936 to Louwet et al. describes photovoltaic devices and polymer materials.

Fundamental organic reactions useful in synthesis herein can be found in for example Advanced Organic Chemistry, 5th Ed, by Smith, March, 2001.

Another descriptive text for organic semiconductors and processing same is Printed Organic and Molecular Electronics, Ed. Gamota et al., 2004.

### Inherently Conductive and Conjugated Polymers

Inherently conductive polymers or conjugated polymers are organic polymers that, due to their conjugated backbone structure, show relatively high electrical conductivities under some conditions (relative to those of traditional polymeric materials). Performance of these materials as a conductor of holes or electrons is increased when they are oxidized or reduced. Upon low oxidation (or reduction) of inherently conductive polymers, in a process which is frequently referred to as doping, an electron is removed from the top of the valence band (or added to the bottom of the conduction band) creating a radical cation (or polaron). Formation of a polaron creates a partial delocalization over several monomeric units. Upon further oxidation, another electron can be removed from a separate polymer segment, thus yielding two independent polarons. Alternatively, the unpaired electron can be removed to create a dication (or bipolaron). In an applied electric field, both polarons and bipolarons are mobile and can move along the polymer chain by delocalization of double and single bonds. This change in oxidation state results in the formation of new energy states, called bipolarons. The energy levels are accessible to some of the remaining electrons in the valence band, allowing the polymer to function as a conductor. The extent of this conjugated structure is dependent upon the polymer chains to form a planar conformation in the solid state. This is because conjugation from ring-to-ring is dependent upon pi-orbital overlap. If a particular ring is twisted out of planarity, the overlap cannot occur and the conjugation band structure can be disrupted. Some minor twisting is not detrimental since the degree of overlap between rings varies as the cosine of the dihedral angle between them.

Performance of a conjugated polymer as an organic conductor can also be dependant upon the morphology of the polymer in the solid state. Electronic properties can be dependent upon the electrical connectivity and inter-chain charge transport between polymer chains. Pathways for charge transport can be along a polymer chain or between adjacent chains. Transport along a chain can be facilitated by a planar backbone conformation due to the dependence of the charge carrying moiety on the amount of double-bond character between the rings, an indicator of ring planarity. This conduction mechanism between chains can involve either a stacking of planar, polymer segment, called pi-stacking, or an inter-chain hopping mechanism in which excitons or electrons can tunnel or “hop” through space or other matrix to another chain that is in proximity to the one that it is leaving. Therefore, a process that can drive ordering of polymer chains in the solid state can help to improve the performance of the conducting polymer. It is known that the absorbance characteristics of thin films of inherently conductive polymers reflect the increased re-stacking which occurs in the solid state.

In applications such as polymer-based solar cells, polymer light emitting diodes, organic transistors, or other organic circuitry the flow of electrons and positive conductors (i.e. “holes”) is dictated by the relative energy gradient of the conduction and valence bands within the components. Therefore, suitable materials of preferred embodiments for a given application are selected for the values of their energy band levels which may be suitably approximated through analysis of ionization potential (as measured by cyclic voltammetry) Micaroni, L et al., J. Solid State Electrochem., 2002, 7, 55-59 and references sited therein) and band gap (as determined by UV/Vis/NIR spectroscopy as described in Richard D. McCullough, Adv. Mater., 1998, 10, No. 2, pages 93-116, and references cited therein).

In one embodiment, the conjugated polymer comprises a homopolymer, a copolymer, a terpolymer, a random copolymer, a block copolymer, or an alternating copolymer. Suitable inherently conductive polymers include, but are not limited to, poly(thiophene), poly(thiophene) derivatives, poly(pyrrole), poly(pyrrole) derivatives, poly(aniline), poly(aniline) derivatives, poly(phenylene vinylene), poly(phenylene vinylene) derivatives, poly(thienylene vinylene), poly(thienylene vinylene) derivatives, poly(bis-thienylene vinylene), poly(bis-thienylene vinylene) derivatives, poly(acetylene), poly(acetylene) derivatives, poly(fluorene), poly(fluorene) derivatives, poly(arylene), poly(arylene) derivatives, poly(isothianaphthalene), poly(isothianaphthalene) derivatives, and mixtures thereof.

In some embodiments, suitable inherently conductive polymer has a molecular weight of from, for example, about 1,000 to about 40,000 g/mol. In certain cases, suitable inherently conductive polymers have a molecular weight of, for example, from about 1000; 10,000; or 20,000 to about 30,000 or 40,000 g/mol. The polymer can have a number average molecular weight of at least about 2,000 g/mol, or at least about 5,000 g/mol, or at least about 20,000 g/mol. Molecular weight can be measured by, for example, gel permeation chromatography using, for example, chloroform as eluent and applying calibration based on molecular weight standards such as for example polystyrene standards for determination of molecular weight.

Inherently conductive polymers, including methods of making, are described in for example T. A. Skotheim, Handbook of Conducting Polymers, 3^{rd} Ed. (two vol), 2007; Meijer et al., Materials Science and Engineering, 32 (2001), 1-40; and Kim, Pure Appl. Chem., 74, 11, 2031-2044, 2002, and references cited in each of these references.

In particular, polythiophenes are known in the art. They can be homopolymers or copolymers, including block copolymers. They can be soluble. They can be regioregular. In particular, alkoxy- and alkyl-substituted polythiophenes can be used. In particular, regioregular polythiophenes can be used as described in for example U.S. Pat. Nos. 6,602,974 and 6,166,172 to McCullough et al., as well as McCullough, R. D.; Tristram-Nagle, S.; Williams, S. P.; Lowe, R. D.; Jayaraman, M. J. Am. Chem. Soc. 1993, 115, 4910. See also Plextronics (Pittsburgh, Pa.) commercial products. Soluble alkyl- and alkoxy-substituted polymers and copolymers can be used including poly(3-hexylthiophene). For example, the substituent can have five to 20 carbon atoms, or six to 15 carbon atoms. The substitutent can have for example one to five heteroatoms like oxygen, nitrogen, or sulfur. Other examples can be found in U.S. Pat. Nos. 5,294,372 and 5,401,537 to Kochem et al. U.S. Pat. Nos. 6,454,880 and 5,331,183 further describe active layers.

Soluble materials or well dispersed materials can be used in the stack to facilitate processing.

Additional examples of p-type materials can be found in WO 2007/011739 (Gaudiana et al.) which describes polymers having monomers which are substituted cyclopentadithiophene moieties, and which is hereby incorporated by reference in its entirety.

Since overall photovoltaic efficiency of inherently conductive polymers can be limited by insufficient solar absorptivity, the absorptivity of the polymeric system can be enhanced by covalently linking chromophores onto the polymer backbone. Accordingly, porphyrinic macrocycle compounds can be added to inherently conductive polymers to enhance solar absorptivity.

### Structures of Porphyrinic Materials and Macrocycles

The term “porphyrinic macrocycle” refers to a porphyrin or porphyrin derivative. Such derivatives include porphyrins with extra rings ortho-fused, or ortho-perifused, to the porphyrin nucleus, porphyrins having a replacement of one or more carbon atoms of the porphyrin ring by an atom of another element (skeletal replacement), derivatives having a replacement of a nitrogen atom of the porphyrin ring by an atom of another element (skeletal replacement of nitrogen), derivatives having substituents other than hydrogen located at the peripheral (meso-, beta-) or core atoms of the porphyrin, derivatives with saturation of one or more bonds of the porphyrin (hydroporphyrins, e.g., chlorins, bacteriochlorins, isobacteriochlorins, decahydroporphyrins, corphins, pyrrocorphins, etc.), derivatives obtained by coordination of one or more metals to one or more porphyrin atoms (metalloporphyrins), derivatives having one or more atoms, including pyrrolic and pyrromethenyl units, inserted in the porphyrin ring (expanded porphyrins), derivatives having one or more groups removed from the porphyrin ring (contracted porphyrins, e.g., corrin, corrole) and combinations of the foregoing derivatives (e.g phthalocyanines, porphyrazines, naphthalocyanines, subphthalocyanines, and porphyrin isomers). Preferred porphyrinic macrocycles comprise at least one 5-membered ring.

The term “porphyrin” refers to a cyclic structure typically composed of four pyrrole rings together with four nitrogen atoms and two replaceable hydrogens for which various metal atoms can readily be substituted. A typical porphyrin is hemin.

A “chlorin” is essentially the same as a porphyrin, but differs from a porphyrin in having one partially saturated pyrrole ring. The basic chromophore of chlorophyll, the green pigment of plant photosynthesis, is a chlorin.

A “bacteriochlorin” is essentially the same as a porphyrin, but differs from a porphyrin in having two partially saturated non-adjacent (i.e., trans) pyrrole rings.

An “isobacteriochlorin” is essentially the same as a porphyrin, but differs from a porphyrin in having two partially saturated adjacent (i.e., cis) pyrrole rings.

Organization of synthetic porphyrinic pigments into solid-state arrays can afford improvements in solar absorptivity, excitonic energy transduction, charge separation, and electron transfer in organic photovoltaic (OPV) devices. The porphyrinic macrocycle compounds (e.g., porphyrins, chlorins, bacteriochlorins) can exhibit intense absorption in the blue, red, and near-infrared (NIR, 700-900 nm) region.

Advances in synthetic capabilities can enable tunability of electronic structure (including, for instance, control of molecular orbital energy levels and energy gap), incorporation of reactive groups for macroscale organization, and control over solubility to enable low-cost device fabrication procedures.

Porphyrinic macrocycles for starting materials in preferred embodiments can be synthesized by methods presented in, for example, U.S. Pat. Nos. 6,849,730; 6,603,070; 6,916,982; 6,559,374; 6,765,092; and 6,946,552, herein incorporated by reference in their entirety. Additional references include, for example:

### Methods of Making Porphyrins

“Refined Synthesis of 5-Substituted Dipyrromethanes,” Littler, B. J.; Miller, M. A.; Hung, C.-H.; Wagner, R. W.; O'Shea, D. F.; Boyle, P. D.; Lindsey, J. S. J. Org. Chem. 1999, 64, 1391-1396.
“Synthesis of meso-Substituted Porphyrins,” Lindsey, J. S. In The Porphyrin Handbook; Kadish, K. M., Smith, K. M., Guilard, R., Eds.; Academic Press: San Diego, Calif., 2000; Vol. 1, pp 45-118.
“Efficient Synthesis of Monoacyl Dipyrromethanes and Their Use in the Preparation of Sterically Unhindered trans-Porphyrins,” Rao, P. D.; Littler, B. J.; Geier, G. R., III; Lindsey, J. S. J. Org. Chem. 2000, 65, 1084-1092.
“Rational Syntheses of Porphyrins Bearing up to Four Different Meso Substituents,” Rao, P. D.; Dhanalekshmi, S.; Littler, B. J.; Lindsey, J. S. J. Org. Chem. 2000, 65, 7323-7344.
“A Survey of Acid Catalysts in Dipyrromethanecarbinol Condensations Leading to meso-Substituted Porphyrins,” Geier, G. R., III; Callinan, J. B.; Rao, P. D.; Lindsey, J. S. J. Porphyrins Phthalocyanines 2001, 5, 810-823.
“A Scalable Synthesis of Meso-Substituted Dipyrromethanes,” Laha, J. K.; Dhanalekshmi, S.; Taniguchi, M.; Ambroise, A.; Lindsey, J. S. Org. Process Res. Dev. 2003, 7, 799-812.
“A Tin-Complexation Strategy for Use with Diverse Acylation Methods in the Preparation of 1,9-Diacyldipyrromethanes,” Tamaru, S.-I.; Yu, L.; Youngblood, W. J.; Muthukumaran, K.; Taniguchi, M.; Lindsey, J. S. J. Org. Chem. 2004, 69, 765-777.
“Boron-Complexation Strategy for Use with 1-Acyldipyrromethanes,” Muthukumaran, K.; Ptaszek, M.; Noll, B.; Scheidt, W. R.; Lindsey, J. S. J. Org. Chem. 2004, 69, 5354-5364.
“9-Acylation of 1-Acyldipyrromethanes Containing a Dialkylboron Mask for the α-Acylpyrrole Motif,” Zaidi, S. H. H.; Muthukumaran, K.; Tamaru, S.-I.; Lindsey, J. S. J. Org. Chem. 2004, 69, 8356-8365.
“Direct Synthesis of Palladium Porphyrins from Acyldipyrromethanes,” Sharada, D. S.; Muresan, A. Z.; Muthukumaran, K.; Lindsey, J. S. J. Org. Chem. 2005, 70, 3500-3510.
“1,9-Bis(N,N-dimethylaminomethyl)dipyrromethanes in the Synthesis of Porphyrins Bearing One or Two Meso Substituents,” Fan, D.; Taniguchi, M.; Yao, Z.; Dhanalekshmi, S.; Lindsey, J. S. Tetrahedron 2005, 61, 10291-10302.
“Imine-Substituted Dipyrromethanes in the Synthesis of Porphyrins Bearing One or Two Meso Substituents,” Taniguchi, M.; Balakumar, A.; Fan, D.; McDowell, B. E.; Lindsey, J. S. J. Porphyrins Phthalocyanines 2005, 9, 554-574.
“Alkylthio Unit as an a-Pyrrole Protecting Group for use in Dipyrromethane Synthesis,” Thamyongkit, P.; Bhise, A. D.; Taniguchi, M.; Lindsey, J. S. J. Org. Chem. 2006, 71, 903-910.
“Investigation of Streamlined Syntheses of Porphyrins Bearing Distinct Meso Substituents,” Zaidi, S. H. H.; Fico, R., Jr.; Lindsey, J. S. Org. Process Res. Dev. 2006, 10, 118-134.

### Methods of Making Chlorins

“Rational Synthesis of Meso-Substituted Chlorin Building Blocks,” Strachan, J.-P.; O'Shea, D. F.; Balasubramanian, T.; Lindsey, J. S. J. Org. Chem. 2000, 65, 3160-3172. Additions and Corrections: Strachan, J.-P.; O'Shea, D. F.; Balasubramanian, T.; Lindsey, J. S. J. Org. Chem. 2001, 66, 642.
“Rational Synthesis of b-Substituted Chlorin Building Blocks,” Balasubramanian, T.; Strachan, J. P.; Boyle, P. D.; Lindsey, J. S. J. Org. Chem. 2000, 65, 7919-7929.
“Synthesis of meso-Substituted Chlorins via Tetrahydrobilene-a Intermediates,” Taniguchi, M.; Ra, D.; Mo, G.; Balasubramanian, T.; Lindsey, J. S. J. Org. Chem. 2001, 66, 7342-7354.
“Synthesis and Electronic Properties of Regioisomerically Pure Oxochlorins,” Taniguchi, M.; Kim, H.-J.; Ra, D.; Schwartz, J. K.; Kirmaier, C.; Hindin, E.; Diers, J. R.; Prathapan, S.; Bocian, D. F.; Holten, D.; Lindsey, J. S. J. Org. Chem. 2002, 67, 7329-7342.
“Introduction of a Third Meso Substituent into Diaryl Chlorins and Oxochlorins,” Taniguchi, M.; Kim, M. N.; Ra, D.; Lindsey, J. S. J. Org. Chem. 2005, 70, 275-285.
“Refined Synthesis of 2,3,4,5-Tetrahydro-1,3,3-trimethyldipyrrin, a Deceptively Simple Precursor to Hydroporphyrins,” Ptaszek, M.; Bhaumik, J.; Kim, H.-J.; Taniguchi, M.; Lindsey, J. S. Org. Process Res. Dev. 2005, 9, 651-659.
“Synthetic Chlorins Bearing Auxochromes at the 3- and/or 13-Positions,” Laha, J. K.; Muthiah, C.; Taniguchi, M.; McDowell, B. E.; Ptaszek, M.; Lindsey, J. S. J. Org. Chem. 2006, 71, 4092-4102.
“A New Route for Installing the Isocyclic Ring in Chlorins Yielding 131-Oxophorbines,” Laha, J. K.; Muthiah, C.; Taniguchi, M.; Lindsey, J. S. J. Org. Chem. 2006, 71, 7049-7052.
“Sparsely Substituted Chlorins as Core Constructs in Chlorophyll Analogue Chemistry. Part 1: Synthesis,” Ptaszek, M.; McDowell, B. E.; Taniguchi, M.; Kim, H.-J.; Lindsey, J. S. Tetrahedron 2007, 63, 3826-3839.
“Sparsely Substituted Chlorins as Core Constructs in Chlorophyll Analogue Chemistry. Part 2: Derivatization,” Taniguchi, M.; Ptaszek, M.; McDowell, B. E.; Lindsey, J. S. Tetrahedron 2007, 63, 3840-3849.
“Sparsely Substituted Chlorins as Core Constructs in Chlorophyll Analogue Chemistry. Part 3: Spectral and Structural Properties,” Taniguchi, M.; Ptaszek, M.; McDowell, B. E.; Boyle, P. D.; Lindsey, J. S. Tetrahedron 2007, 63, 3850-3863.
   Methods of Making Bacteriochlorins “De Novo Synthesis of Stable Tetrahydroporphyrinic Macrocycles: Bacteriochlorins and a Tetradehydrocorrin,” Kim, H.-J.; Lindsey, J. S. J. Org. Chem. 2005, 70, 5475-5486.

Porphyrinic macrocycle compounds can be bonded or linked covalently to inherently conductive polymers to provide advantages. Since overall photovoltaic efficiency of inherently conductive polymers is limited by insufficient solar absorptivity, the absorptivity of the polymeric system can be enhanced by covalently linking porphyrin macrocyclic units onto the polymer backbone, side groups, or end groups.

An additional molecular chromophore can increase the total absorption and afford a direct increase in external quantum efficiency of OPV cells versus control cells lacking the additional chromophore. Molecular architectures with well-defined absorption and hole transport properties can improve the bulk heterojunction between the n- and p-type materials resulting in improved photovoltaic efficiencies. As polymers that absorb further in the red are developed, the porphyrin sensitizers can be replaced or augmented with chlorins or even bacteriochlorins. The bandgap of the polymer can be less than that of the excited-state energy of the porphyrinic photosensitizer to facilitate energy transfer will occur. This criterion can be satisfied with porphyrinic macrocycles.

Representative molecular design considerations are that (1) energy transfer occurs from photoexcited porphyrinic macrocycle to the polymer, either intramolecularly to the attached polymer, or intermolecularly to polymer in close proximity, without competing electron-transfer quenching, (2) the porphyrinic macrocycle does not serve as a hole trap (i.e., is not readily oxidized), and (3) the appropriate phase segregation of the resulting porphyrin macrocyle-polymer and n-type component, such as fullerene, still occurs. The oxidation potential of the porphyrinic macrocycle can be tuned by nearly 1V by appropriate choice of substituents and metal on the porphyrinic macrocycle. The substituents described herein can provide a combination of variation in oxidation potential and steric encumbrance. Steric encumbrance can be tuned to tailor solubility and distance of approach of neighboring polymer chains.

The substituents on the porphyrinic macrocycle can be chosen to tailor energetics and solubility. For example, in the porphyrin macrocyclic family the electrochemical potential of a given porphyrin can be tuned over quite a wide range by incorporation of electron-withdrawing or electron-releasing substituents (Yang, S. I. et al., J. Porphyrins Phthalocyanines 1999, 3, 117-147). Examples of such substituents include aryl, phenyl, cycloalkyl, alkyl, halogen, alkoxy, alkylthio, perfluoroalkyl, perfluoroaryl, pyridyl, cyano, thiocyanato, nitro, amino, N-alkylamino, acyl, sulfoxyl, sulfonyl, imido, amido, and carbamoyl.

In one embodiment, the metal is present. In another embodiment, the metal is not present (metal-free).

When present, the central metal of the porphyrinic macrocycle can also be chosen to tailor energetics. With monomeric porphyrinic macrocycle, variation in electrochemical potential can be obtained with different central metals (Fuhrhop, J.-H.; Mauzerall, D. J. Am. Chem. Soc. 1969, 91, 4174-4181). A wide variety of metals can be incorporated in porphyrinic macrocycles. Those metals that are photochemically active include, but are not limited to, Zn, Mg, Al, Sn, Cd, Au, Pd, and Pt. Counterions can be present. Porphyrins generally form very stable radical cations (Felton, R. H. In The Porphyrins; Dolphin, D., Ed.; Academic Press: New York, 1978; Vol. V, pp 53-126).

One approach for preparing the conjugates entails reaction of a suitably substituted-porphyrin and a suitably substituted-conductive polymer. One embodiment provides a composition comprising at least one conjugated polymer, at least one porphyrinic macrocycle, wherein the conjugated polymer and porphyrinic macrocycle are bonded or covalently linked to each other. Bonding can be covalent, ionic, dative in character. The conjugated polymer and porphyrinic macrocycle may be bonded directly to one another or they may be bonded through a linker or spacer group.

The attachment can be carried out on intact polymers which are singly or doubly end-capped. The attachment can be through covalent bonding including use of linker groups to link together the porphyrinic macrocycle and conjugated polymer. The porphyrinic macrocycle can be bonded to an end group of the conducting polymer or a side group of the conducting polymer.

Porphyrinic macrocycles can be attached to one, two, three, four, or more polymer chains. In certain cases, the porphyrinic macrocycles can be attached to one or two polymer chains.

Also, the inherently conductive polymer can have from about 1 to about 200 bonds to porphyrinic macrocycles. In certain cases, the inherently conductive polymer has from about 2 to about 100 bonds to porphyrinic macrocycles. In certain cases, the inherently conductive polymer has from about 10 to about 50 bonds to porphyrinic macrocycles.

A suitably-substituted porphyrinic macrocycle and a conductive polymer whose backbone comprises a functional group that is able to react with the suitably-substituted porphyrinic macrocycle can be combined. Many reactions are available to perform coupling between a porphyrinic macrocycle and a conductive polymer. Depending on the type of reaction, the porphyrinic macrocycle and the conductive polymer, proper substituents on the compounds facilitate the coupling.

A suitable coupling reaction to combine a porphyrinic macrocycle with a conductive polymer can be nucleophilic substitution. Examples of reactants for a nucleophilic substitution include compounds with leaving groups such as halo, mesylate, tosylate, haloalkyl, and compounds with nucleophilic groups such as hydroxyl, amino, thiol, hydroxylalkyl, aminoalkyl, and thioalkyl. The halo can be for example bromo.

Thus, a porphyrinic macrocycle can comprise a leaving group and a conductive polymer can comprise a nucleophilic group. In certain cases, when a porphyrinic macrocycle comprises a leaving group, at least one of S¹, S², S³, S⁴, S⁵, S⁶, S⁷, S⁸, S⁹, S¹⁰, S¹¹, S¹², S¹³, S¹⁴, S¹⁵, and S¹⁶ of a compound of formula Ia, Ib, and Ic is a leaving group or comprises a leaving group.

In another embodiment, a porphyrinic macrocycle can comprise a nucleophilic group and a conductive polymer can comprise a leaving group. In certain cases, when a porphyrinic macrocycle comprises a leaving group, at least one of S¹, S², S³, S⁴, S⁵, S⁶, S⁷, S⁸, S⁹, S¹⁰, S¹¹, S¹², S¹³, S¹⁴, S¹⁵, and S¹⁶ of a compound of formula Ia, Ib, and Ic is a nucleophilic group or comprises a leaving group.

In one embodiment, the inherently conductive polymer has bonds to the porphyrinic macrocycle derived from or through a functional group on the conductive polymer backbone selected from halo, hydroxyl, amino, thiol, boronic acid, boronate, acyl, formyl, acetyl, carboxylic acid, carboxylate ester, haloalkyl, hydroxylalkyl, aminoalkyl, and thioalkyl.

Non-covalent linkages can encompass a variety of molecular interactions including van der Waals forces, hydrogen bonding, and electrostatic forces. The latter include salt formation between ionizable groups. Typical hydrogen-bonding groups include amides, imides, sulfonamides, alcohols with ketones, and the like.

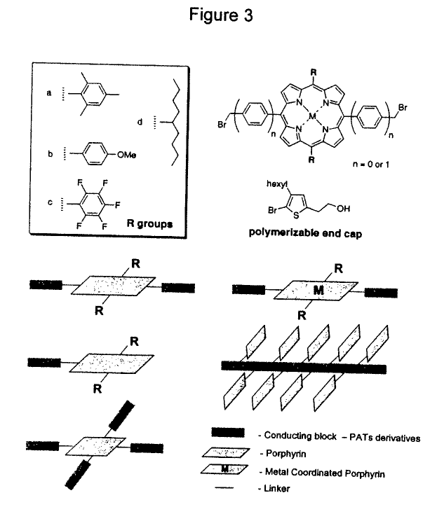

FIG. 3, which is merely illustrative, shows an example of a coupling reaction of a porphyrinic macrocycle and polymer utilizing nucleophilic substitution. In FIG. 3, a bromomethyl-substituted porphyrinic macrocycle and a hydroxyalkylthiophene are used to prepare a porphyrinic macrocycle-polymer conjugate. Alteration of the number of bromomethyl groups on the porphyrinic macrocycle results in corresponding designs with attachment to variable number of polymer chains.

Other suitable coupling reactions include organometallic-mediated coupling reactions (e.g., Suzuki coupling, Stille coupling, Heck coupling, Hartwig-Buchwald coupling, Kumada coupling) and more traditional coupling procedures (e.g., Wittig reaction, Williamson ether synthesis), and the like.

Embodiments provide for a composition comprising a soluble, inherently conductive polymer with at least one bond to a porphyrinic macrocycle through L, wherein
the porphyrinic macrocycle comprising a chemical entity selected from Formula Ia, Ib, Ic,

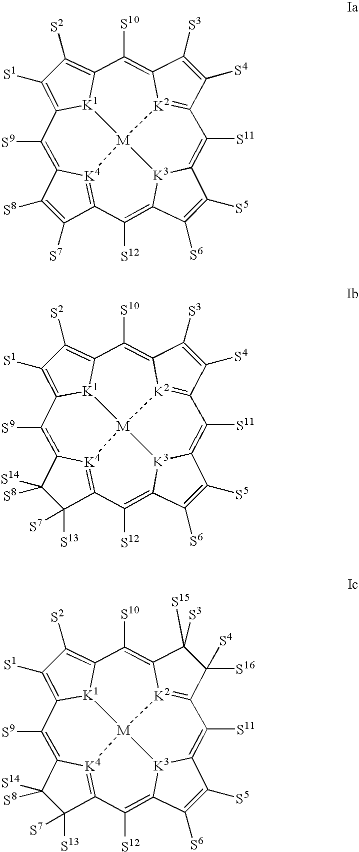

wherein:
M, when present, is selected from Zn, Mg, Pt, Pd, Sn, Ni, Si, Al, Au, and Ag;
K¹, K², K³ and K⁴ are each independently selected from Se, NH, CH₂, O, and S; S¹, S², S³, S⁴, S⁵, S⁶, S⁷, S⁸, S⁹, S¹⁰, S¹¹, S¹², S¹³, S¹⁴, S¹⁵, and S¹⁶ are each independently selected from hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, acyl, formyl, carboxylic acid, acylamino, ester, amide, imide, hydroxyl, nitro, alkylthio, amino, alkylamino, arylalkylamino, disubstituted amino, acyloxy, sulfoxyl, sulfonyl, sulfonate, sulfonamide, thiocyanato, urea, alkoxylacylamino, aminoacyloxy, and L-Y;
wherein each pair of S⁸ and S¹⁴, S⁷ and S¹³, S³ and S¹⁵, or S⁴ and S¹⁶, can together form ═O;
wherein each of S⁸ and S¹⁴, S⁷ and S¹³, S³ and S¹⁵, or S⁴ and S¹⁶, can together form spiroalkyl;
wherein each pair of S¹ and S², S³ and S⁴, S⁵ and S⁶, and S⁷ and S⁸, can together form an annulated arene, which annulated arene is unsubstituted or substituted one or more time with a substituent selected from hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, acyl, formyl, carboxylic acid, acylamino, ester, amide, imide, hydroxyl, nitro, alkylthio, amino, alkylamino, arylalkylamino, disubstituted amino, acyloxy, sulfoxyl, sulfonyl, sulfonate, sulfonamide, thiocyanato, urea, alkoxylacylamino, aminoacyloxy, and L-Y;
each L is selected from a bond, alkylene, alkyleneoxy, arylene, heterocyclylene, heteroarylene, alkylarylene, alkenylene, and alkynylene;
each Y is selected from hydrogen, the inherently conductive polymer, or a second porphyrinic macrocycle selected from Formula Ia, Ib, and Ic; and
   wherein said composition is capable of harvesting energy.

In formulas Ia, Ib, and Ic, the metal M can be removed as appropriate.

Synthesis can be also carried out so that a spacer group is used to link the porphyrinic macrocycle to the polymer. The spacer group can be based on a carbon chain or a carbon chain also comprising one or more heteroatoms. One or more repeat units can be present. Mixtures of repeat units can be present. For example, spacer group can comprise alkylene or alkyleneoxy units, or propyleneoxy units, including for example spacer groups with two to 50 carbon atoms, or three to 25 carbon atoms.

Additional embodiments are provided below.

### Electrochemical and Spectroscopic Studies of Molecules and Multicomponent Architectures

Detailed physico-chemical studies can be performed on the porphyrinic building blocks and various multicomponent architectures, including the porphyrinic macrocycle-polymer conjugated materials. Studies include, for example: (1) static absorption and emission spectroscopies; (2) resonance Raman spectroscopy; (3) electron paramagnetic resonance (EPR) spectroscopy (of paramagnetic species); (4) x-ray photoelectron (XPS) and infrared (IR) spectroscopy of surface-bound species; (5) electrochemical measurements; and (6) density functional theory (DFT) calculations. These studies aid in understanding mechanisms and commercial applications of energy flow by, for example, evaluating: (1) the electronic structure of the individual molecules; (2) the changes that occur in these properties upon incorporation into multicomponent architectures; and (3) how attachment to the surface affects the properties of the multicomponent architectures.

### Porphyrinic Macrocycle/Polymer Conjugate Cells

One structure to utilize directed energy flow is active layers based on porphyrinic macrocycle/polymer conjugates. These materials can be characterized in sandwich cells where the active layer comprises: (1) the active porphyrinic macrocycle/polymer conjugate; and (2) mixed nanoscale morphologies including phase segregated polymer/fullerene blends. This cell takes advantage of the high molecular absorptivity of the porphyrinic macrocycle, and enables excitons produced upon light absorption from the porphyrinic macrocycle to be directed to the polymer. An energy diagram of this system can be described. The absorptivity of a wider gap porphyrinic macrocycle is larger than the polymer at the same energy, increasing the net absorbance. On the energy diagram, the wider gap material is shown behind the polymer to indicate that it serves primarily as a pathway for photon capture and exciton delivery to the polymer, which then makes contacts to the electron acceptor and anode electrodes (as in a typical bilayer cell). Combining high absorptivity porphyrinic monomers with lower gap polymers to achieve broader solar spectral coverage can be used for future low cost polymer cells.

### Device Fabrication

Devices using the presently claimed inventions can be made using for example indium tin oxide (ITO) as an anode material on a substrate. Other anode materials can include for example metals, such as Au, carbon nanotubes, single or multiwalled, and other transparent conducting oxides. The resistivity of the anode can be maintained below for example 15 Ω/sq or less, 25 or less, 50 or less, or 100 or less. The substrate can be for example glass, plastics (PTFE, polysiloxanes, thermoplastics, PET, PEN and the like), metals (Al, Au, Ag), metal foils, metal oxides, (TiOx, ZnOx) and semiconductors, such as Si. The ITO on the substrate can be cleaned using techniques known in the art prior to device layer deposition. An optional hole injection layer (HIL) and/or hole transport layer (HTL) can be added using for example spin casting, ink jetting, doctor blading, spray casting, dip coating, vapor depositing, or any other known deposition method. The HIL can be for example poly(3,4-ethylenedioxythiophene) (PEDOT), poly(3,4-ethylenedioxythiophene)poly(styrenesulfonate) PEDOT/PSS or N,N′-diphenyl-N,N′-bis(3-methylphenyl)-1,1′-biphenyl-4,4′-diamine (TBD), or N,N′-diphenyl-N,N′-bis(1-napthylphenyl)-1,1′-biphenyl-4,4′-diamine (NPB), or Plexcore HIL (Plextronics, Pittsburgh, Pa.).

The thickness of the HIL layer can be for example from 10 nm to 300 nm thick, or from 30 nm to 60 nm, or 60 nm to 100 nm, or 100 nm to 200 nm. The film then can be optionally dried/annealed at 110 to 200° C. for 1 min to an hour, optionally in an inert atmosphere.

The active layer can be formulated from a mixture of n-type and p-type materials. The n- and p-type materials can be mixed in a ratio of for example from about 0.1 to 4.0 (p-type) to about 1 (n-type) based on a weight, or from about 1.1 to about 3.0 (p-type) to about 1 (n-type) or from about 1.1 to about 1.5 (p-type) to about 1 (n-type). The amount of each type of material or the ratio between the two types of components can be varied for the particular application.

The n- and p-type materials can be mixed in a solvent at for example from about 0.01 to about 0.1% volume solids. The solvents useful for the presently claimed inventions can include, for example, halogenated benzenes, alkyl benzenes, halogenated methane, and thiophenes derivatives, and the like. More specifically, solvent can be for example chlorobenzene, dichlorobenzene, xylenes, toluene, chloroform, and mixtures thereof.

The active layer can be then deposited by spin casting, ink jetting, doctor blading, spray casting, dip coating, vapor depositing, or any other known deposition method, on top of the HIL film. The film is then optionally annealed at for example about 40 to about 250° C., or from about 150 to 180° C., for about 10 min to an hour in an inert atmosphere.

Next, a cathode layer can be added to the device, generally using for example thermal evaporation of one or more metals. For example, a 1 to 15 nm Ca layer is thermally evaporated onto the active layer through a shadow mask, followed by deposition of a 10 to 300 nm Al layer.

In some embodiments an optional interlayer may be included between the active layer and the cathode, and/or between the HTL and the active layer. This interlayer can be for example from 0.5 nm to about 100 nm, or from about 1 to 3 nm, thick. The interlayer can comprise an electron conditioning, a hole blocking, or an extraction material such as LiF, BCP, bathocuprine, fullerenes or fullerene derivatives, such as C60 and other fullerenes and fullerene derivatives discussed herein.

The devices can be then encapsulated using a glass cover slip sealed with a curable glue, or in other epoxy or plastic coatings. Cavity glass with a getter/desiccant may also be used.

In addition, the active layer can comprise additional ingredients including for example surfactants, dispersants, and oxygen and water scavengers.

The active layer can comprise multiple layers or be multi-layered.

The active layer composition can comprise a mixture in the form of a film.

Electrodes, including anodes and cathodes, are known in the art for photovoltaic devices. Known electrode materials can be used. Transparent conductive oxides can be used. Transparency can be adapted for a particular application. For example, the anode can be indium tin oxide, including ITO supported on a substrate. Substrates can be rigid or flexible.

### Active Layer Morphology

The active layer can form a P/N composite including nanoscale phase separated structures and bulk heterojunction. See for example discussion of nanoscale phase separation in bulk heterojunctions in Dennler et al., “Flexible Conjugated Polymer-Based Plastic Solar Cells: From Basics to Applications,” Proceedings of the IEEE, vol. 93, no. 8, August 2005, 1429-1439. Conditions and materials can be selected to provide for good film formation, low roughness (e.g., 1 nm RMS), and discrete, observable, phase separation characteristics can be achieved. The present invention can have phase separated domains on a scale of about 5 to 50 nm as measured by AEM. AFM analysis can be used to measure surface roughness and phase behavior. In general, phase separated domains are not desirable so that both donor and acceptor are uniformly and continuously distributed in the active layer.

See also Belcher et al., Solar Energy Materials & Solar Cells, 91 (2007) 447-452 (“The effect of porphyrin inclusion on the spectral response of ternary P3HT:porphyrin:PCBM bulk heterojunction solar cells.”).

### Blend Embodiment

One alternative embodiment provides a composition comprising a blend of: at least one conjugated polymer, wherein the conjugated polymer comprises a polythiophene, at least one porphyrinic macrocycle, wherein the conjugated polymer and the porphyrinic macrocycle are not bonded to each other. The composition can further comprise an n-acceptor such as for example a fullerene or a fullerene derivative.

In this embodiment, for example, the conjugated polymer can be a regioregular polythiophene. In addition, the conjugated polymer can be for example a 3-substituted polythiophene.

The weight ratio of the porphyrinic macrocycle to the polythiophene can be for example between about 1:5 and 1:15, or about 1:7 to about 1:13.

The porphyrinic macrocycle can be metal free or can comprise a metal. The metal can be for example zinc or any of the other metals noted above.

Films can be prepared from solvent mixtures and solutions.

The blend composition in for example film form can be annealed.

In another embodiment, the blend composition is not annealed and application of heat is avoided.

Another embodiment provides a composition comprising a blend of: at least one p-type semiconductor and at least one additive which absorbs in the UV and IR outside of the absorption region of the semiconductor. p-Type semiconductors are known in the art and can be organic or inorganic. They can be polymeric. In one embodiment, the semiconductor is a conjugated polymer and the additive is a porphyrinic macrocycle, as described above.

### Device Performance

Known solar cell parameters can be measured including for example J_{SC} (mA/cm²) and Voc (V) and fill factor and efficiency (%) by methods known in the art.

The efficiency can be at least 3.5%, or at least 4%, or at least 4.5%, or at least 5.0%, or at least 5.5%, or at least 6.0%.

The fill factor can be at least about 0.60, or at least about 0.63, or at least about 0.67.

The Voc (V) can be at least about 0.56, or at least about 0.63, or at least about 0.82.

The Jsc (mA/cm²) can be at least about 8.92, or at least about 9.20, or at least about 9.48.

The efficiency can be measured for a device comprising an active layer comprising a fullerene adduct and the efficiency compared to efficiency for a control including a substantially analogous device but wherein the active layer comprises poly(3-hexylthiophene)-PCBM.

An increase in efficiency can be determined and measured to be at least about 5%, or at least about 10%, or at least about 15%, or at least about 20%, or at least about 25%, or at least about 30%, or at least about 35%, or at least about 40%, or at least about 45%, or at least about 50%.

### Methods of Making and Using

Methods are also providing for making the devices and using the devices. Known methods for printing, depositing, and patterning layers can be used including solution or vacuum based methods.

Ink compositions can be formulated comprising a carrier or solvent system for the polymer and when present the n-acceptor.

The following examples are provided to illustrate certain aspects of the present invention and to aid those of skill in the art in practicing the invention. These examples are in no way to be considered to limit the scope of the invention.

### Additional Embodiments

SYNTHESIS: Functionalizing P3HT Polymers with Porphyrins

In five additional embodiments, two types of representative polymers such as P3HT polymers can be used: a Br—OH terminated polymer (P1) and an H—Br terminated polymer (P2). The end groups shown in P1 and P2 can be reversed, as complex mixtures of chains and end groups may be present which may vary in different embodiments. In addition, in some cases where Br is shown as an end group, H may also be present as an end group in substantial amounts; in some cases, the Br may be present in a majority amount relative to H end groups.

One first embodiment comprises end-capping P1 (hydroxyl and bromine end groups) with suitably functionalized porphyrins. See for example Scheme 1.

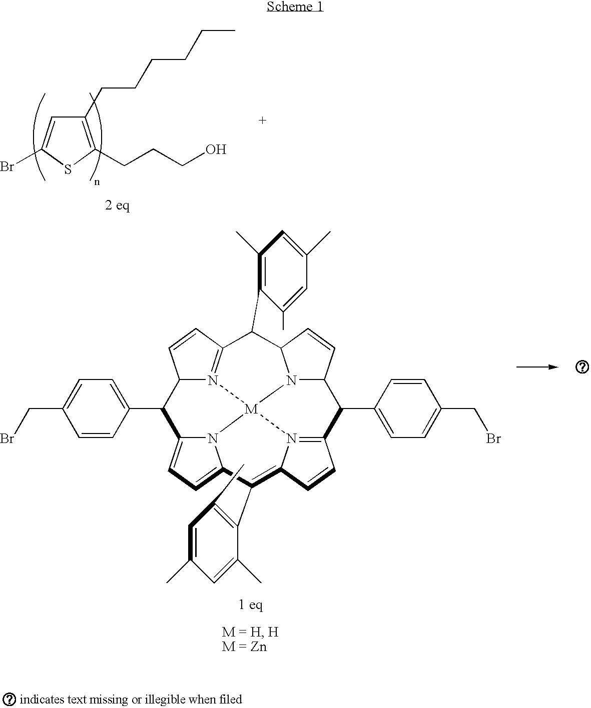

Another second embodiment comprises end-capping the polymer P1 (hydroxyl and bromine end groups) with an aryl aldehyde, which in turn could be used as a precursor to a porphyrin. (Scheme 2).

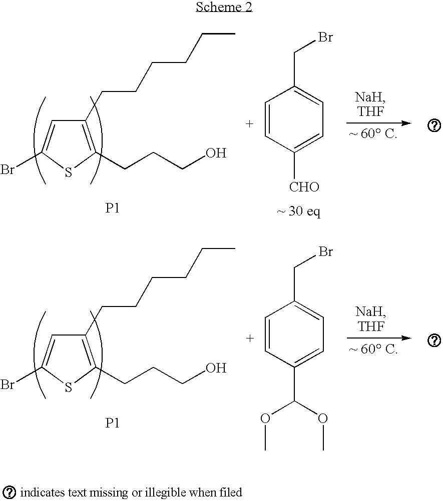

In another third embodiment, the Sonogashira coupling of 5,15-bis(4-ethynylphenyl)-10,20-dimesitylporphyrin and P1 can be carried out using the standard conditions of, for example, Pd₂(dba)₃ and P(o-tol)₃ in toluene/TEA (Scheme 3). An example of this embodiment is further provided in the working examples.

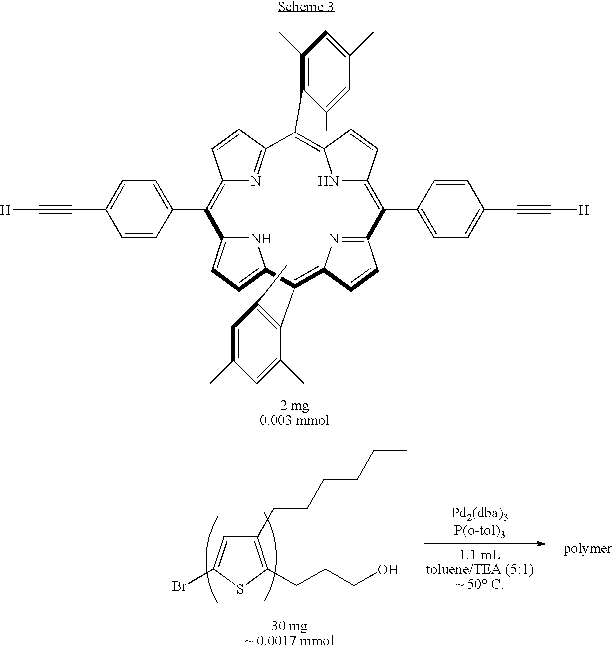

An additional fourth embodiment comprises the Suzuki coupling of polymer P1 and 5,15-bis(3,5-di-tert-butylphenyl)-10-mesityl-20-[4-(4,4,5,5-tetramethyl-1,3,2-dioxaborolan-2-yl]phenyl]porphyrin (excess of porphyrin) which can be carried in toluene/DMF using Pd(PPh₃)₄ as a catalyst (Scheme 4).

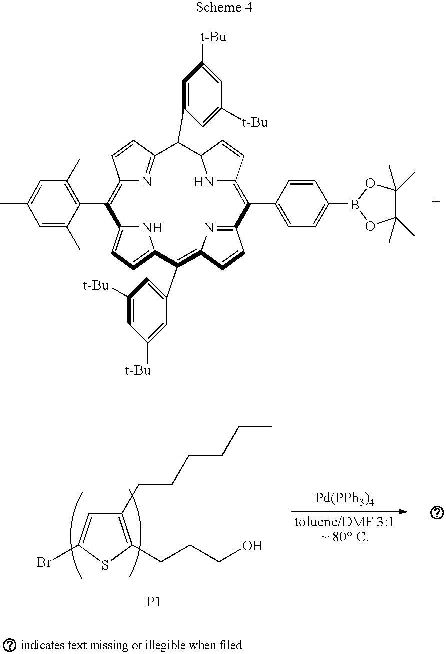

Another fifth embodiment comprises use of H—Br terminated polymer P2 (Scheme 5). The polymer P2 may also comprise polymer chains which have an H—H end group system. In other words, mixtures of end groups can be present such as for example a mixture of H—Br and H—H end groups, with H—Br predominating. The Sonogashira coupling reaction can be performed in the presence of Pd₂(dba)₃ (30 mol %) and P(o-tol)₃ in toluene/triethylamine (5:1). 5,15-Bis(4-ethynylphenyl)-10,20-dimesitylporphyrin can be used as the porphyrin building block. The working examples provide an additional embodiment.

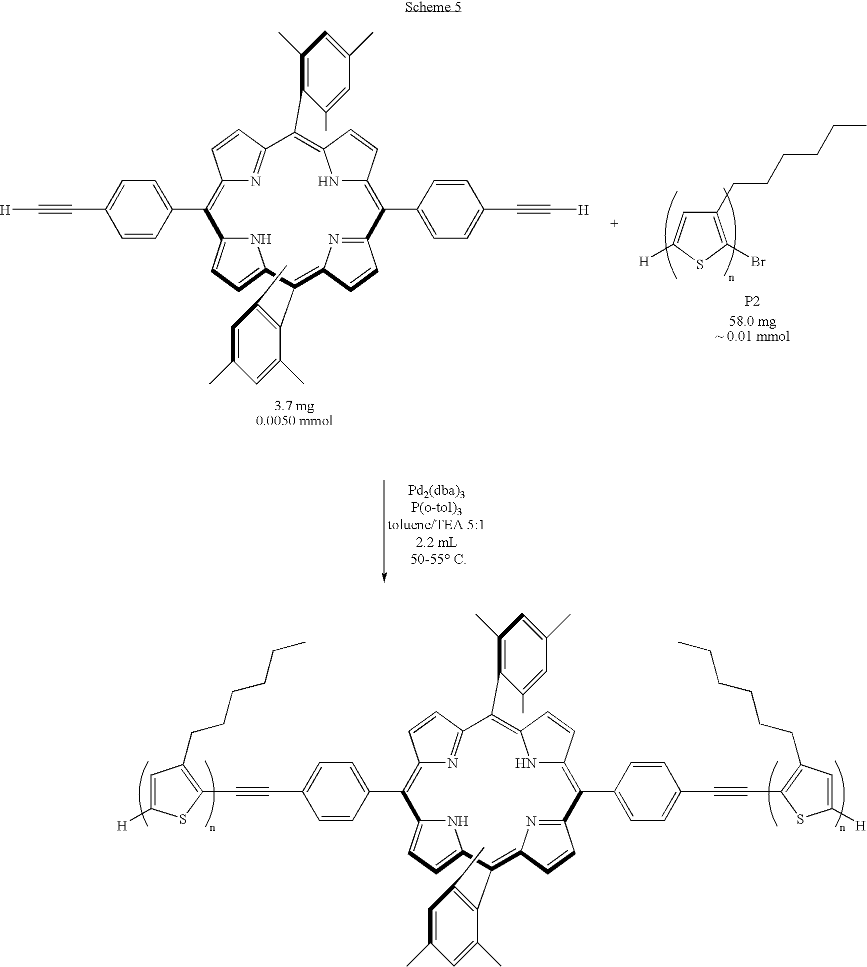

In these and other embodiments, reaction conditions including, for example, reaction duration, temperature, and concentration can be adjusted and adapted.

Additional embodiments and working examples are provided.

### WORKING EXAMPLES

### Example 1

### Scheme 3

In this example, the Sonogashira coupling of 5,15-bis(4-ethynylphenyl)-10,20-dimesitylporphyrin and P1 was carried out using the standard conditions of Pd₂(dba)₃ and P(o-tol)₃ in toluene/TEA (Scheme 3). The conditions employed were typical of those for Sonogashira reactions with porphyrins, where limited solubility requires dilute solution (1.6 mM, 50° C.) (see reference 2). Analytical SEC-HPLC was chosen for monitoring the progress of the reactions. Two new peaks with shorter retention time than that of the starting polymer were observed by SEC-HPLC. The presence of those peaks suggested formation of two new products with molecular weight larger than that of the starting polymer. MALDI-MS analysis after reaction showed two characteristic peaks: one with m/z similar to that of the starting polymer and a second with average m/z two times greater. A part of the crude reaction mixture was chromatographed (preparative SEC column, THF). The ¹H NMR spectrum of the second fraction showed the presence of the polymer and the porphyrin in a ˜1:1 ratio. Thus, the reaction afforded a mixture of the porphyrin containing two attached polymer units and the porphyrin with attached one polymer unit (note also that starting polymer and the starting porphyrin were observed in crude reaction mixture). The reaction conditions are described more below in the Experimental Section (Reaction 1).

### Example 2

### Scheme 5

Another embodiment comprises use of H—Br terminated polymer P2 (Scheme 5). The Sonogashira coupling reaction was performed in the presence of Pd₂(dba)₃ (30 mol %) and P(o-tol)₃ in toluene/triethylamine (5:1). 5,15-Bis(4-ethynylphenyl)-10,20-dimesitylporphyrin was chosen as the porphyrin building block.

After initial trials, the ratio of porphyrin/P2 was set as ˜1:2. The reaction was monitored by analytical SEC-HPLC. After 16 h, SEC analysis showed complete consumption of the starting porphyrin, formation of two new products (t_{R}=31.2 min and 32.5 min, with the ratio ˜2:3) and a peak corresponding to the starting polymer (t_{R}=34.7 min). The ratio of the total sum of the products to the unreacted starting material (˜3:1) corresponds to the ratio of Br—H/H—H terminated polymers in the starting material (4:1). The crude mixture was therefore filtered through Celite, precipitated from CHCl₃ and used for further studies without additional purification. The ¹H NMR spectra of the resulting mixture showed the presence of the porphyrin (in the ratio of polymer/porphyrin>2:1); however, LD-MS analysis showed only m/z corresponding to the starting polymer. The reaction conditions are described more in the Experimental Section (Reaction 2).

The analytical SEC data clearly show the presence of higher molecular weight species formed upon reaction. See FIG. 4. The absorption spectrum of the crude reaction mixture, as well as the product obtained from preparative SEC separation, shows the presence of both the polymer and the porphyrin. The absorption spectrum of each component eluting in advance of the P3HT polymer (upper panel) upon analytical SEC characterization showed the absorption peaks of both the porphyrin and the polymer. See FIG. 5. These data provide very strong support of the presence of porphyrin-polymer conjugates.

### Experimental Section

Measurements.

¹H spectra were recorded on a Bruker Avance AV-300 (operating at 300.13 MHz in ¹H), Bruker Avance DMX-500 (operating at 500.13 MHz in ¹H) spectrometers. Some ¹H NMR spectra were recorded on a spectrometer operating at 400 MHz. All NMR spectra were recorded in deuterated chloroform (CDCl₃) as solvent containing 0.003% TMS as an internal reference unless noted otherwise.

Absorption spectra were collected in dichloromethane at room temperature.

MALDI-TOF MS was performed using a Voyager-DE STR BioSpectrometry Workstation by PerSeptive Biosystems. All spectra were recorded using 2,2′:5′,2″-terthiophene (Aldrich) as a matrix in the linear ion mode, in which samples were irradiated under high vacuum (<10⁻⁶ torr) using a nitrogen laser (wavelength 337 nm, 2 ns pulse). The accelerating voltage was 24 kV. Grid voltage and low mass gate were 85.0% and 500.0 Da, respectively.

Gel Permeation Chromatography (GPC) traces for end-functionalized poly(3-hexylthiophene) were recorded on a Waters 2690 Separations Module apparatus and a Waters 2487 Dual λ Absorbance Detector where chloroform was the eluent (flow rate 1.0 mL/min, 35° C., λ=254 nm) with a series of three Styragel columns (10⁵, 10³, 100 Å; Polymer Standard Services) and a guard column. Calibration based on polystyrene standards purchased from Polymer Standard Service was applied for determination of molecular weights and toluene was used as an internal standard.

PLgel columns (50, 100 and 500 Å, THF) were used for analytical SEC. Furthermore, for H—Br capped P3HT five columns ((50, 100 and 500 Å, and two 1000 Å) were also used. Bio-Beads S-X1 (40-80 μm) from Bio-Rad were used for preparative SEC column chromatography.

Materials for synthesis of poly(3-hexylthiophene). All reactions were conducted under prepurified nitrogen, using either flame-dried or oven-dried glassware. All glassware was assembled while hot and cooled under nitrogen. Commercial chemicals, e.g., [1,3-bis(diphenylphosphino)propane]dichloronickel(II) (Ni(dppp)Cl₂), Grignard reagents were purchased from Aldrich Chemical Co., Inc. and used without further purification. Prior to use, tetrahydrofuran (THF) was dried over and distilled from sodium benzophenone ketyl. Titration of the Grignard reagents was performed following the procedure described by Love (Love, B. E.; Jones, E. G. J. Org. Chem. 1999, 64, 3755).

Poly(3-hexylthiophene) (P3HT) polymers P1 and P2 terminated with OH/Br aid H/Br, respectively, were synthesized according to published literature procedures (Ref. (3) and (4) and (5)) utilizing the Grignard Metathesis (GRIM) method (Scheme 1). The polymers were characterized by ¹H NMR and GPC. The average molecular weights of P1 and P2 were M_{w}=17,100 (PDI=1.1 (GPC)) and M_{w}=12,600 (PDI=1.1 (GPC)), respectively. MALDI-TOF MS was utilized to monitor the end-group composition of the polymers, where P1 contained a mixture of OH/Br and OH/H termini (with ˜60:40% mixture of Br to H ratio) and P2 was predominately terminated with Br (80:20% mixture of Br to H ratio) (Ref 1).

One synthesis for P1 is further illustrated:

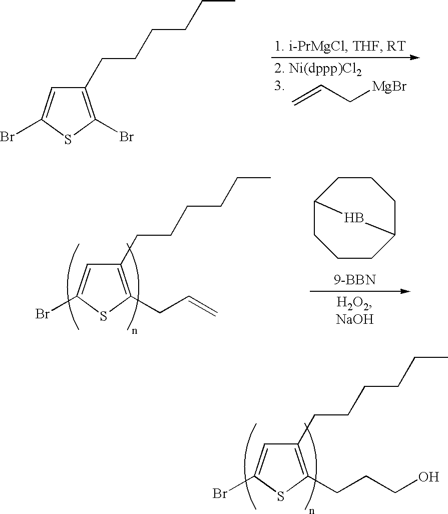

Reaction 1. Samples of polymer P1 (30 mg, 0.0018 mmol), 5,15-bis(4-ethynylphenyl)-10,20-dimesitylporphyrin (2 mg, 0.003 mmol, 3.3 mM), Pd(PPh₃)₄ (0.5 mg, 0.5 μmol), and [P(o-tol)₃] (1.3 mg, 0.0042 mmol) were placed into a 10 mL Schlenk flask. The flask was pump-purged with argon three times. Toluene/TEA (1.1 mL, 5:1) was added, and the mixture was stirred at 50° C. under argon for 20 h. The following observations were made:
HPLC—suggests the presence of two compounds with higher molecular weight than starting polymer (HPLC; t_{R}=18.1 min, 18.4 and 19.0 min); the first peak has t_{R}˜1 min shorter than the retention time of the starting polymer (t_{R}=19.1 min). Part of the crude reaction mixture was chromatographed (SEC preparative column, THF). Four fractions were collected. The absorption spectrum was recorded for each fraction. The data and datafiles are as follows:

### Fraction 1 “SNGSH_F1” λ (CH₂Cl₂) 451, 607, 656 nm

### Fraction “SNGSH_F2” λ (CH₂Cl₂) 422, 455 nm

### Fraction “SNGSH_F2” λ (CH₂Cl₂) 423 455 nm

### Fraction “SNGSH_F4” λ (CH₂Cl₂) 423, 555 604 nm

The ¹H NMR spectrum and the MALDI-MS spectrum were recorded for the first and for the second fraction. The ¹H NMR spectrum of the second fraction suggests the presence of polymer and porphyrin in a 1:1 ratio.
The HPLC was measured for each fraction.

Reaction 2. Samples of P2 (58.0 mg, 0.00997 mmol), 5,15-bis(4-ethynylphenyl)-10,20-dimesitylporphyrin (3.7 mg, 0.0050 mmol), Pd₂(dba)₃ (1.4 mg, 0.0014 mmol) and P(o-tol)₃ (3.2 mg, 0.010 mmol) were placed in a Schienk flask, and pump-purged with argon (5 times). A degassed mixture of toluene/triethylamine (2.2 mL) was added, and the resulting mixture was stirred at 55° C. for 16 h (note that P2 was not soluble at room temperature, but dissolved completely at 55° C.). The reaction mixture was diluted with CHCl₃ (˜50 mL), filtered through Celite, and the filtrate was concentrated. The resulting product was dissolved in THF and chromatographed on preparative SEC column (to remove putative traces of unreacted porphyrin). The fractions containing polymer were collected, concentrated, dissolved in CHCl₃, precipitated with MeOH, filtered and dried under high vacuum to afford a brown solid (50 mg).

### Device Testing:

Device testing showed that photosensitization by porphyrins was observed in blends as well as in chemically-bonded P3HT and porphyrin. Photocurrent from porphyrins was demonstrated.

Device testing also showed that in some embodiments heat treatment had negative effects in blends of porphyrins and P3HT.

Device testing also showed that photosensitization was observed in chemically-bonded porphyrin and P3HT even after heat treatment. While the present embodiments are not limited by theory, it may be that chemical bonding does not allow porphyrin to diffuse at annealing temperatures.

EQE analysis showed that the porphyrinic system can achieve about 13% bonus in quantum efficiency. Normalization of this to AM1.5G reference solar spectrum reveals potential porphyrinic contribution to cell photocurrent is about 3%, consistent with absorbance modeling.

### REFERENCES

(1) Jeffries-E L, et al. Macromolecules 2005, 38, 1034610352.
(2) Loewe, R. S., et al. J. Mater. Chem. 2002, 12, 1530-1552.
(3) Iovu, M., et al. Macromolecule, 2005, 38, 8649-8656.
(4) Iovu, M., et al. Polymer, 2005, 46, 8582-8586.
(5) Jeffries-E L, et al. Adv. Mater., 2004, 16, 1017-1019.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A composition comprising:
at least one polymer comprising at least one porphyrinic macrocycle bonded to at least one conjugated polymer, wherein the porphyrinic macrocycle is metal-free.

2. The composition according to claim 1, wherein the conjugated polymer comprises solubilizing side groups.

3. The composition according to claim 1, wherein the conjugated polymer comprises at least one polythiophene, polyaniline, polypyrrole, polyphenylene vinylene, polyfluorene, polyphenylene, poly(thienylene vinylene), poly(bis-thienylene vinylene), poly(acetylene), poly(arylene), poly(isothianaphthalene), and combinations thereof.

4. The composition according to claim 1, wherein the conjugated polymer comprises at least one polythiophene.

5. The composition according to claim 1, wherein the conjugated polymer comprises at least one 3-substituted polythiophene.

6. The composition according to claim 1, wherein the conjugated polymer comprises at least one regioregular polythiophene.

7. The composition according to claim 1, wherein the conjugated polymer comprises a homopolymer, a copolymer, a terpolymer, a random copolymer, a block copolymer, or an alternating copolymer.

8. The composition according to claim 1, wherein the porphyrinic macrocycle comprises a porphyrin, a chlorin, or a bacteriochlorin.

9. The composition according to claim 1, wherein the porphyrinic macrocycle comprises a chlorin or a bacteriochlorin.

10. The composition according to claim 1, wherein the porphyrinic macrocycle comprises a moiety represented by Formula Ia, Ib, Ic, or combinations thereof: 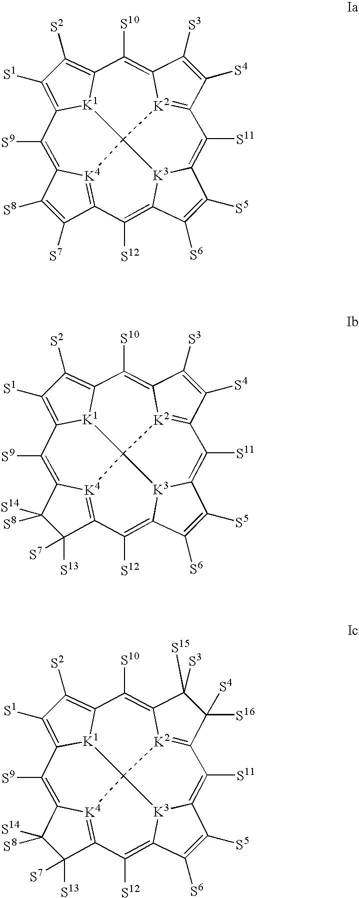
wherein:
K¹, K², K³ and K⁴ are each independently selected from Se, NH, CH₂, O, and S;
S¹, S², S³, S⁴, S⁵, S⁶, S⁷, S⁸, S⁹, S¹⁰, S¹¹, S¹², S¹³, S¹⁴, S¹⁵, and S¹⁶ are each independently selected from hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, acyl, formyl, carboxylic acid, acylamino, ester, amide, imide, hydroxyl, nitro, alkylthio, amino, alkylamino, arylalkylamino, disubstituted amino, acyloxy, sulfoxyl, sulfonyl, sulfonate, sulfonamide, thiocyanato, urea, alkoxylacylamino, and aminoacyloxy;
wherein each pair of S⁸ and S¹⁴, S⁷ and S¹³, S³ and S¹⁵, or S⁴ and S¹⁶, can together form ═O;
wherein each of S⁸ and S¹⁴, S⁷ and S¹³, S³ and S¹⁵, or S⁴ and S¹⁶, can together form spiroalkyl;
wherein each pair of S¹ and S², S³ and S⁴, S⁵ and S⁶, and S⁷ and S⁸, can together form an annulated arene, which annulated arene is unsubstituted or substituted one or more time with a substituent selected from hydrogen alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, acyl, formyl, carboxylic acid, acylamino, ester, amide, imide, hydroxyl, nitro, alkylthio, amino, alkylamino, arylalkylamino, disubstituted amino, acyloxy, sulfoxyl, sulfonyl, sulfonate, sulfonamide, thiocyanato, urea, alkoxylacylamino, aminoacyloxy.

11. The composition according to claim 1, wherein the porphyrinic macrocycle is linked to the polymer by a spacer group.

12. The composition according to claim 1, wherein the porphyrinic macrocycle is bonded to an end group of the conjugated polymer.

13. The composition according to claim 1, wherein the porphyrinic macrocycle is bonded to a side group of the conjugated polymer.

14. The composition according to claim 1, wherein one porphyrinic macrocycle is bonded to one chain of the conjugated polymer.

15. The composition according to claim 1, wherein one porphyrinic macrocycle is bonded to at least two chains of the conjugated polymer.

16. The composition according to claim 1, wherein one porphyrinic macrocycle is bonded to four chains of the conjugated polymer.

17. The composition according to claim 1, wherein the porphyrinic macrocycle and the polymer are linked by an ester linkage, an amide linkage, an ether linkage, a urethane linkage, an amino linkage, a thioether linkage, or a thioester linkage.

18. The composition according to claim 1, further comprising an n-type acceptor material.

19. The composition according to claim 1, further comprising a fullerene or a fullerene derivative.

20. The composition according to claim 1, wherein the composition is a soluble composition.

21. The composition according to claim 1, wherein the polymer has a number average molecular weight of at least about 2,000 g/mol.

22. The composition according to claim 1, wherein the polymer has a number average molecular weight of at least about 5,000 g/mol.

23. The composition according to claim 1, wherein the polymer has a number average molecular weight of at least about 20,000 g/mol.

24. The composition according to claim 1, wherein the polymer comprises a backbone comprising at least ten conjugated repeat units.

25. The composition according to claim 1, wherein the polymer comprises at least two different porphyrinic macrocycles different from each other and each bonded to at least one conjugated polymer.

26. The composition according to claim 1, wherein the conjugated polymer comprises a polythiophene derivative, and the porphyrin is bonded to the conjugated polymer by a spacer group.

27. The composition according to claim 1, wherein the conjugated polymer comprises a polythiophene derivative, and wherein the porphyrinic macrocycle is bonded to an end group of the conjugated polymer.

28. The composition according to claim 1, wherein the conjugated polymer comprises a polythiophene derivative, and wherein the porphyrinic macrocycle is bonded to a side group of the conjugated polymer.

29. The composition according to claim 1, wherein the conjugated polymer comprises a polythiophene derivative, and wherein the porphyrinic macrocycle is bonded to an end group of the conjugated polymer.

30. The composition according to claim 1, wherein the conjugated polymer comprises a regioregular polythiophene derivative, and wherein the porphyrinic macrocycle is bonded to a side group or an end group of the conducting polymer, and the composition further comprises an n-acceptor.

31. A composition comprising:
at least one polymer comprising at least one porphyrinic macrocycle covalently linked to at least one conjugated polymer, wherein the conjugated polymer has at least 10 conjugated repeat units.

32. A composition according to claim 31, wherein the conjugated polymer comprises a polythiophene.

33. A composition according to claim 31, wherein the conjugated polymer comprises a regioregular polythiophene.

34. A composition according to claim 31, wherein the conjugated polymer comprises solubilizing side groups.

35. A composition according to claim 31, wherein the porphyrinic macrocycle comprises metal.

36. A composition according to claim 31, wherein the porphyrinic macrocycle is metal-free.

37. A composition according to claim 31, wherein the porphyrinic macrocycle comprises porphyrin, chlorin, or bacteriochlorin.

38. A composition according to claim 31, wherein the composition further comprises an n-acceptor.

39. A composition according to claim 31, wherein the composition further comprises a fullerene or fullerene derivative.

40. A composition according to claim 31, wherein the porphyrinic macrocycle is bonded to at least two chains of conjugated polymer.

41. A composition prepared by:
providing at least one porphyrinic macrocycle,
providing at least one conjugated polymer,
covalently linking the conjugated polymer and the porphyrinic macrocycle.

42. A method comprising:
providing at least one porphyrinic macrocycle,
providing at least one conjugated polymer,
covalently linking the conjugated polymer and the porphyrinic macrocycle.

43. An ink composition comprising the composition according to claim 1.

44. A solid state electronic device comprising a
a first electrode,
a second electrode,
an active layer disposed between the first and second electrodes, wherein the active layer comprises a composition according to claim 1.

45. The device according to claim 44, wherein the device comprises a solar cell.

46. The device according to claim 44, wherein the device further comprises a hole injection layer between one electrode and the active layer.

47. The device according to claim 44, wherein the device further comprises a polythiophene hole injection layer between one electrode and the active layer.

48. The device according to claim 44, wherein the device comprises an organic light emitting diode.

49. The device according to claim 44, wherein the device comprises an organic light emitting diode, and wherein the device further comprises a hole injection layer between one electrode and the active layer

50. The device according to claim 44, wherein the active layer further comprises a fullerene or fullerene derivative.

51. A composition comprising a blend of:
at least one conjugated polymer, wherein the conjugated polymer comprises a polythiophene,
at least one porphyrinic macrocycle, wherein the conjugated polymer and the porphyrinic macrocycle are not covalently bonded to each other.

52. The composition according to claim 51, wherein the conjugated polymer is a regioregular polythiophene.

53. The composition according to claim 51, wherein the conjugated polymer is a 3-substituted polythiophene.

54. The composition according to claim 51, the composition further comprising an n-acceptor.

55. The composition according to claim 51, the composition further comprising a fullerene or a fullerene derivative.

56. A composition comprising:
at least one polymer comprising at least one porphyrinic macrocycle bonded to at least one conjugated polymer, wherein the porphyrinic macrocycle comprises a bacteriochlorin or a chlorine.

57. The composition according to claim 56, wherein the porphyrinic macrocycle comprises a metal.

58. The composition according to claim 56, wherein the porphyrinic macrocycle is free of metal.

59. A composition comprising a blend of:
at least one p-type semiconductor and at least one additive which absorbs in the UV and IR outside of the absorption region of the semiconductor.

60. The composition of claim 59, wherein the semiconductor is a conjugated polymer and the additive is a porphyrinic macrocycle.
